(19) United States
(12) Patent Application Publication
(10) Pub. No.: US 2012/0088714 A1
(43) Pub. Date: Apr. 12, 2012

(54) SYSTEM FOR STORAGE AND SUBSEQUENT HANDLING OF BOTULINUM TOXIN

(75) Inventors: Harish PM Kumar, Fullerton, CA (US); Orest Olejnik, Coto de Caza, CA (US)

(73) Assignee: Allergan, Inc., Irvine, CA (US)

(21) Appl. No.: 13/267,798

(22) Filed: Oct. 6, 2011

Related U.S. Application Data
(60) Provisional application No. 61/390,546, filed on Oct. 6, 2010.

(51) Int. Cl. A61K 38/02; B65D 85/00

(52) U.S. Cl. 514/1.1; 206/524.1

(57) ABSTRACT

A system and method for storage of botulinum toxin containing pharmaceutical compositions is herein disclosed. Particular aspects of the instant disclosure relate to vials having preferred internal geometries that provide optimized lyophilization, vacuum drying, storage, reconstitution and extraction of a botulinum toxin-containing pharmaceutical composition.

### CROSS-REFERENCE TO RELATED APPLICATIONS

This application claims the benefit of U.S. Provisional Patent Application Ser. No. 61/390,546, filed on Oct. 6, 2010, the entire disclosure of which is incorporated herein by this specific reference.

### FIELD

The present disclosure relates to systems for the storage and subsequent handling of pharmaceutical compositions. More particularly and in one aspect, a system is provided for optimized lyophilization, vacuum drying, storage, reconstitution, and extraction of a botulinum toxin-containing pharmaceutical composition from a storage vessel, such as, for example, a vial, or the like.

### BACKGROUND

Safe and effective drug therapy by injection depends not only upon accurate diagnosis, but also on efficient and reliable introduction of the medical substance into the subcutaneous cellular tissue without introducing contaminants or ambient air. The applicable drug or pharmaceutical must first be drawn from the resident container or vial into a syringe before injection. The integrity and features of the vial therefore affect the overall safety of the injection.

The bacterium Clostridium botulinum produces botulinum toxin (BTX), a neurotoxic protein. There are different types of botulinum toxin, such as, for example, botulinum toxin type A (“BTX-A”) and botulinum toxin type B (“BTX-B”). As an example, BTX-A containing pharmaceutical products include BOTOX® (botulinum toxin type A complex, human serum albumin, and sodium chloride), DYSPORT® (botulinum toxin type A complex, human serum albumin, and lactose) and XEOMIN® (neurotoxic component of botulinum toxin type A, human serum albumin, and sucrose), which are all provided in a form requiring reconstitution. An example of a BTX-B containing pharmaceutical product is MYOBLOC® (botulinum toxin type B, human serum albumin, sodium succinates, and sodium chloride) which is provided in solution form and thus is not reconstituted prior to administration.

Typically, BTX products on the market are packaged in vials with a flat or slightly-concave bottom. For example, BOTOX® is packaged in a vial typically referred to as the “squat vial,” which includes a flat or slightly-concave bottom. As discussed above, BTX products may be supplied as a solution or as a solid. For example, BOTOX® is supplied as a vacuum-dried composition which includes BTX-A, human serum albumin, and sodium chloride. When supplied as a solid, BTX products usually require reconstitution before being withdrawn from the vial.

Presently, maximizing extraction of BTX compositions from vials is a concern for medical practioners. Once reconstituted, or when provided in liquid form, a portion of the BTX composition often remains in vials following extraction for a number of reasons. For example, BTX powder resulting from the fragmenting of vacuum-dried or lyophilized BTX can adhere to the walls of the storage vial and thus not be reconstituted. Similarly, at times liquid BTX composition (pre-vacuum dried or pre-lyophilized) placed in the vial can dry in more than one location within the storage vial. Further, in certain cases a practitioner may employ an angle of extraction of the BTX composition that is not optimal. In any case, when a portion of a BTX-containing composition remains in its storage vial following extraction, a doctor's estimation of a patient's unit dose can be inaccurate, and of additional concern is the waste of an expensive drug. Because of these issues, persons qualified to administer BTX-A, for example, have attempted manipulations such as tilting or inverting the vials during extraction, or removing the crimp seal and stopper from vials prior to extraction. See Dykstra, D., et al., “Maximizing Extraction of Botulinum Toxin Type A From Vials,” Arch Phys Med Rehabil., vol. 83, pp. 1638-1640. November 2002. However, among other disadvantages, these manipulations are inconvenient and open-vial extraction creates opportunities for contamination.

Thus, there is a need for a system by which botulinum toxin-containing compositions are disposed into storage vessels that will provide improved methods for lyophilization and vacuum drying (if they are to be dried and stored for later reconstitution), as well provide for improved and more complete extraction of the medicament from the vial by an end user.

### BRIEF DESCRIPTION OF THE DRAWINGS

FIG. 1 is an exterior view of one embodiment of the invention.

FIG. 2 is a longitudinal cross-section of the interior of an embodiment of the invention wherein the well has an angular shape and flat bottom.

FIG. 3A is a longitudinal cross-section of the interior of an embodiment of the invention wherein the well with a cylindrical shape and a rounded bottom resembles a “U” shape in the cross-sectional view.

FIG. 3B is a top-down view of an embodiment of the “collecting-well vial,” wherein the well has a cylindrical shape and a rounded bottom.

FIG. 4A is a longitudinal cross-section of the interior of an embodiment of the invention wherein the well with a conical shape resembles a “V” shape in the cross-sectional view.

FIG. 4B is a longitudinal cross-section of the interior of an embodiment of the invention wherein the well with a cubicle shape resembles a square, rectangle, or trapezoid in the cross-sectional view.

FIG. 4C is a longitudinal cross-section view of the interior of an embodiment of the invention wherein the well is not situated in the middle of the interior bottom of the vial.

FIG. 5 is an exterior view (through the transparent material of the vial) of the interior of an embodiment of the invention wherein a needle is inserted through the elastomer material covering the mouth opening of the vial and the needle's tip is placed into the well.

FIG. 6 is an exterior view (through the transparent material of the vial) of the interior of an embodiment of the invention wherein the well with a conical shape resembles a “V” shape in the cross-sectional view.

### DESCRIPTION

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained through use of embodiments of the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value or range of values falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate embodiments of the invention and does not pose a limitation on the scope of the invention otherwise claimed. No language in the specification should be construed as indicating any non-claimed element essential to the practice of the invention.

As used herein, “about” means plus or minus about ten percent of a number, parameter or characteristic described herein.

“Botulinum toxin” means a neurotoxin produced by Clostridium botulinum, as well as a botulinum toxin (or the light chain or the heavy chain thereof) made recombinantly by a non-Clostridial species. The phrase encompasses the botulinum toxin serotypes A, B, C, D, E, F and G. Botulinum toxin, as used herein, also encompasses both a botulinum toxin complex (i.e. the 300, 600 and 900 kDa complexes) as well as the purified botulinum toxin (i.e. about 150 kDa). Modified botulinum toxins such as those with modified targeting domains are within the scope of “botulinum toxin” as are recombinantly made, hybrid, modified, and chimeric botulinum toxins.

“Purified botulinum toxin” is defined as a botulinum toxin that is isolated, or substantially isolated, from other proteins, including proteins that form a botulinum toxin complex. A purified botulinum toxin may be greater than 95% pure, and preferably is greater than 99% pure.

“Pharmaceutical composition” means a formulation with at least one active ingredient, in which an active ingredient can be a botulinum toxin. The word “formulation” means that there is at least one additional ingredient in the pharmaceutical composition besides a botulinum neurotoxin active ingredient. A pharmaceutical composition is therefore a formulation which is suitable for diagnostic or therapeutic administration (i.e. by intramuscular or subcutaneous injection or by insertion of a depot or implant) to a subject, such as a human patient. The pharmaceutical composition can be: in a lyophilized or vacuum dried condition; a solution formed after reconstitution of the lyophilized or vacuum dried pharmaceutical composition with saline or water, or; as a solution which does not require reconstitution. As stated, a pharmaceutical composition can be liquid or solid, for example vacuum-dried. The constituent ingredients of a pharmaceutical composition can be included in a single composition (that is all the constituent ingredients, except for any required reconstitution fluid, are present at the time of initial compounding of the pharmaceutical composition) or as a two-component system, for example a vacuum-dried composition reconstituted with a diluent such as saline which diluent contains an ingredient not present in the initial compounding of the pharmaceutical composition. A two-component system provides the benefit of allowing incorporation of ingredients which are not sufficiently compatible for long-term shelf storage with the first component of the two component system. For example, the reconstitution vehicle or diluent may include a preservative which provides sufficient protection against microbial growth for the use period, for example one-week of refrigerated storage, but is not present during the two-year freezer storage period during which time it might degrade the toxin. Other ingredients, which may not be compatible with a botulinum toxin or other ingredients for long periods of time, can be incorporated in this manner; that is, added in a second vehicle (i.e. in the reconstitution fluid) at the approximate time of use.

As used herein, “stabilization agent” or “stabilizing agent” means any agent that prolongs the biologic activity, or specifically the neurotoxicity of the botulinum neurotoxin, upon storage. In a preferred embodiment, the stabilization or stabilizing agent is a monosaccharide or disaccharide or protein or polymer.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, any references or printed publications cited herein are individually incorporated by reference in their entirety.

Even further, specific embodiments disclosed herein may be further limited in the claims using consisting of or and consisting essentially of language. When used in the claims, whether as filed or added per amendment, the transition term “consisting of” excludes any element, step, or ingredient not specified

Embodiments of the invention relate to containers which can be sterilized for medical purposes, in particular for the storage of pharmaceutical or diagnostic products, including solutions. In certain embodiments the containers can be glass. In some embodiments such containers are intended to come into direct contact with their contents. A varied selection of glass containers can be used, for example, small bottles (described in detail in, for example, the ISO norm 8362, section 1), ampoules (described in detail in, for example, the ISO norm 9187, section 1), syringe bodies (described in detail in, for example, the ISO norm 11040, section 4), glass cylinders (described in detail in, for example, the ISO norm 13926, section 1), as well as bottles (described in detail in, for example, the ISO norm 8356, section 1). The filling volume of these types of containers varies from 0.5 to 2000 ml.

For these purposes, for example, for the packaging of injection solutions, glasses with a high hydraulic resistance are useful (in accordance with the pharmacopoeia, for example, the German Pharmacopoeia DAB 10, glass of the type I or II). Examples of glass containers which fulfill this demand are disclosed in the German utility model DE 296 09 958.U1 which describes glass containers whose surfaces are in contact with the solutions and are have a coating of oxides and/or nitrides of the elements Si, Ti, Ta, Al by way of a plasma chemical vapor deposition (CVD) procedure.

Certain embodiments of the invention utilize, for example, borosilicate glass of the 1^{st} hydrolytic class which is highly resistant chemically, thermally and has low extractables. In certain embodiments, the borosilicate glass is made from ASTM Type I, Class A, borosilicate 33 expansion glass.

Certain embodiments can comprise amber glass vials made from ASTM Type I, Class B, borosilicate 51 expansion glass. Embodiments of the invention meet all of the requirements for Type I glass as specified in the current revision of the U.S. Pharmacopeia.

For a vast number of medical and pharmaceutical uses, it is necessary to sterilize the empty containers before filling them. In certain embodiments, methods and storage vessels of the invention can be include sterilization. Sterilization methods suitable for glass containers at the moment often involve costly technical chemical procedures such as fumigation with ethylene oxide, autoclaving with overheated water vapor and heat sterilization at temperatures of between 250 and 300 C.

Further methods of sterilization using high energy radiation (for example .beta.-radiation, .gamma.-radiation and strong UV-radiation) are not suitable in this case since current glasses, for example, common borosilicate glasses or soda-lime silica glasses, will discolor heterogeneously yellow to brown, often in a spotty manner after the sterilization due to the high energy radiation, depending on the radiation dose. Such discoloration varies depending on time, temperature and the influence of light. Accordingly, it is frequently impossible to render a dependable visual inspection of the contents.

The examination of, for example, a powdery content, for foreign particles would not be dependable in a heterogeneously (spotty) discolored container. Given the high degree of automation in production lines today, any noticeable deviation from a pre-determined norm would lead to a sorting out of the container in question and could possibly even lead to a halt of an entire production line.

Embodiments of the invention can comprise pharmaceutical compositions suitable for administration to a human or animal for a therapeutic, diagnostic, research or cosmetic purpose. These compositions can comprise an active ingredient. The pharmaceutical composition can also include one or more excipients, buffers, carriers, stabilizers, preservatives and/or bulking agents. The active ingredient in a pharmaceutical composition can be a biologic such as a botulinum toxin. The pharmaceutical compositions disclosed herein can have a pH of between about 5 and 8 when reconstituted or upon injection. In certain embodiments it is preferable that the composition have a pH below 8, such as, for example, 7.9, or 7.8, or 7.7, or 7.6, or 7.5, or 7.4, or 7.3, or 7.2, or 7.1, or 7.0, or 6.9, or 6.8, or 6.7, or 6.6, or 6.5, or 6.4, or 6.3, or 6.2, or 6.1, or 6.0, or 5.9, or 5.8, or 5.7, or 5.6, or 5.5, or 5.4, or 5.3, or 5.2, or 5.1, or the like.

Embodiments of the invention encompass compositions with protein or non-protein stabilizing agents. The protein agents can be, for example, human serum albumin, or gelatin, or the like. The agents can be recombinantly made.

The present invention provides compositions of a botulinum toxin and a stabilizing agent, wherein the agent is selected from the group consisting of: proteins, polymers, lipids and carbohydrates. In a preferred embodiment, the agent is albumin, collagen, epinephrine or hyaluronate. In a more preferred embodiment, the agent is hyaluronate. In the most preferred embodiment, the agent is albumin.

The present invention further provides compositions comprising a botulinum toxin and a stabilizing agent, wherein the agent is an albumin, preferably human serum albumin. Furthermore, in one embodiment, the albumin of the present compositions is recombinantly produced. In one embodiment, the albumin is present in an amount between 550 and 5,500.ug albumin per 100 LD.sub.50 units botulinum toxin. In a further embodiment, albumin is present in an amount between 5,500 and 13,000.ug albumin per 100 LD.sub.50 units botulinum toxin. In a preferred embodiment, albumin is present in an amount between 13,000 and 50,500.ug albumin per 100 LD.sub.50 units botulinum toxin. In a more preferred embodiment, albumin is present in an amount between 50,500 and 505,000.ug albumin per 100 LD.sub.50 units botulinum toxin. In a most preferred embodiment, albumin is formulated as encapsulated microspheres in an amount between 50,500 and 90,500.ug albumin per 100 LD.sub.50 units botulinum toxin.

Preferably, embodiments of the invention can include from about 10 LD.sub.50 units to about 10,000 LD.sub.50 units; or about 20 LD.sub.50 units to about 10,000 LD.sub.50 units; or about 30 LD.sub.50 units to about 10,000 LD.sub.50 units; or about 40 LD.sub.50 units to about 10,000 LD.sub.50 units; or about 50 LD.sub.50 units to about 10,000 LD.sub.50 units; or about 75 LD.sub.50 units to about 10,000 LD.sub.50 units; or about 100 LD.sub.50 units to about 10,000 LD.sub.50 units; or about 150 LD.sub.50 units to about 10,000 LD.sub.50 units; or about 200 LD.sub.50 units to about 10,000 LD.sub.50 units; or about 250 LD.sub.50 units to about 10,000 LD.sub.50 units; or about 300 LD.sub.50 units to about 10,000 LD.sub.50 units; or about 350 LD.sub.50 units to about 10,000 LD.sub.50 units; or about 400 LD.sub.50 units to about 10,000 LD.sub.50 units; or about 450 LD.sub.50 units to about 10,000 LD.sub.50 units; or about 500 LD.sub.50 units to about 10,000 LD.sub.50 units; or about 550 LD.sub.50 units to about 10,000 LD.sub.50 units; or about 600 LD.sub.50 units to about 10,000 LD.sub.50 units; or about 650 LD.sub.50 units to about 10,000 LD.sub.50 units; or about 700 LD.sub.50 units to about 10,000 LD.sub.50 units; or about 750 LD.sub.50 units to about 10,000 LD.sub.50 units; or about 800 LD.sub.50 units to about 10,000 LD.sub.50 units; or about 850 LD.sub.50 units to about 10,000 LD.sub.50 units; or about 900 LD.sub.50 units to about 10,000 LD.sub.50 units; or about 950 LD.sub.50 units to about 10,000 LD.sub.50 units; or about 1000 LD.sub.50 units to about 10,000 LD.sub.50 units; or about 1200 to about 10,000 LD.sub.50 units; or about 1400 LD.sub.50 units to about 10,000 LD.sub.50 units; or about 1600 LD.sub.50 units to about 10,000 LD.sub.50 units; or about 1800 LD.sub.50 units to about 10,000 LD.sub.50 units; or about 2000 LD.sub.50 units to about 10,000 LD.sub.50 units or about 2200 LD.sub.50 units to about 10,000 LD.sub.50 units; or about 2400 LD.sub.50 units to about 10,000 LD.sub.50 units; or about 2500 LD.sub.50 units to about 10,000 LD.sub.50 units.

In some embodiments, embodiments of the invention can comprise from about 1 ng to about 1 microgram, or about 1 ng to about 5 microgram; or about 1 ng to about 10 microgram; or about 1 ng to about 15 microgram; or about 1 ng to about 20 microgram; or about 1 ng to about 25 microgram; or about 1 ng to about 30 microgram; or about 1 ng to about 35 microgram; or about 1 ng to about 40 microgram; or about 1 ng to about 45 microgram; or about 1 ng to about 50 microgram; or about 1 ng to about 60 microgram; or about 1 ng to about 70 microgram; or about 1 ng to about 80 microgram; or about 1 ng to about 90 microgram; or about 1 ng to about 100 microgram; or about 1 ng to about 120 microgram; or about 1 ng to about 140 microgram; or about 1 ng to about 160 microgram; or about 1 ng to about 180 microgram; or about 1 ng to about 200 microgram; or about 1 ng to about 220 microgram; or about 1 ng to about 240 microgram; or about 1 ng to about 260 microgram; or about 1 ng to about 280 microgram; or about 1 ng to about 300 microgram; or about 1 ng to about 320 microgram; or about 1 ng to about 340 microgram; or about 1 ng to about 360 microgram; or about 1 ng to about 380 microgram; or about 1 ng to about 400 microgram; or about 1 ng to about 420 microgram; or about 1 ng to about 440 microgram; or about 1 ng to about 460 microgram; or about 1 ng to about 480 microgram; or about 1 ng to about 500 microgram; or about 1 ng to about 520 microgram; or about 1 ng to about 540 microgram; or about 1 ng to about 560 microgram; or about 1 ng to about 580 microgram; or about 1 ng to about 600 microgram; or about 1 ng to about 620 microgram; or about 1 ng to about 640 microgram; or about 1 ng to 660 microgram; or about 1 ng to about 680 microgram; or about 1 ng to about 700 microgram; or about 1 ng to about 720 microgram; or about 1 ng to about 740 microgram; or about 1 ng to about 760 microgram; or about 1 ng to about 780 microgram; or about 1 ng to about 800 microgram; or about 1 ng to about 820 microgram; or about 1 ng to about 840 microgram; or about 1 ng to about 860 microgram; or about 1 ng to about 880 microgram; or about 1 ng to about 900 microgram; or about 1 ng to about 920 microgram; or about 1 ng to about 940 microgram; or about 1 ng to about 960 microgram; or about 1 ng to about 980 microgram; or about 1 ng to about 1 mg, or about 1 ng to about 5 mg; or about 1 ng to about 10 mg; or about 1 ng to about 15 mg; or about 1 ng to about 20 mg; or about 1 ng to about 25 mg; or about 1 ng to about 30 mg; or about 1 ng to about 35 mg; or about 1 ng to about 40 mg; or about 1 ng to about 45 mg; or about 1 ng to about 50 mg; or about 1 ng to about 60 mg; or about 1 ng to about 70 mg; or about 1 ng to about 80 mg; or about 1 ng to about 90 mg; or about 1 ng to about 100 mg; or about 1 ng to about 120 mg; or about 1 ng to about 140 mg; or about 1 ng to about 160 mg; or about 1 ng to about 180 mg; or about 1 ng to about 200 mg; or about 1 ng to about 220 mg; or about 1 ng to about 240 mg; or about 1 ng to about 260 mg; or about 1 ng to about 280 mg; or about 1 ng to about 300 mg; or about 1 ng to about 320 mg; or about 1 ng to about 340 mg; or about 1 ng to about 360 mg; or about 1 ng to about 380 mg; or about 1 ng to about 400 mg; or about 1 ng to about 420 mg; or about 1 ng to about 440 mg; or about 1 ng to about 460 mg; or about 1 ng to about 480 mg; or about 1 ng to about 500 mg; or about 1 ng to about 520 mg; or about 1 ng to about 540 mg; or about 1 ng to about 560 mg; or about 1 ng to about 580 mg; or about 1 ng to about 600 mg; or about 1 ng to about 620 mg; or about 1 ng to about 640 mg; or about 1 ng to 660 mg; or about 1 ng to about 680 mg; or about 1 ng to about 700 mg; or about 1 ng to about 720 mg; or about 1 ng to about 740 mg; or about 1 ng to about 760 mg; or about 1 ng to about 780 mg; or about 1 ng to about 800 mg; or about 1 ng to about 820 mg; or about 1 ng to about 840 mg; or about 1 ng to about 860 mg; or about 1 ng to about 880 mg; or about 1 ng to about 900 mg; or about 1 ng to about 920 mg; or about 1 ng to about 940 mg; or about 1 ng to about 960 mg; or about 1 ng to about 980 mg; or about 1 ng to or about 1 gram botulinum toxin.

Embodiments of the invention also encompass a pharmaceutical composition comprising (or consisting of or consisting essentially of) a botulinum toxin, and between about 5 grams and about 20 grams of a polyvinylpyrrolidone for each about 100 units of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyvinylpyrrolidone, wherein the potency of the botulinum toxin is at least about 40% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyvinylpyrrolidone, wherein the potency of the botulinum toxin is at least about 50% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a to polyvinylpyrrolidone, and a disaccharide, wherein the potency of the botulinum toxin is at least about 40% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyvinylpyrrolidone, and a disaccharide, wherein the potency of the botulinum toxin is at least about 50% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyvinylpyrrolidone, and a disaccharide, wherein the potency of the botulinum toxin is at least about 60% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyvinylpyrrolidone, and a disaccharide, wherein the potency of the botulinum toxin is at least about 70% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyvinylpyrrolidone, and a polyethylene glycol, wherein the potency of the botulinum toxin is at least about 40% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a compound selected from the group consisting of a first monosaccharide, a first disaccharide, a first trisaccharide, and a first alcohol made by reducing the first monosaccharide, and a compound selected from the group of compounds consisting of a polyethylene glycol, a second monosaccharide, a second disaccharide, a second trisaccharide, a metal, a second alcohol, and an amino acid, wherein the second monosaccharide, the second disaccharide and the second trisaccharide are different from respectively the first monosaccharide, the first disaccharide, and the first trisaccharide, wherein the potency of the botulinum toxin at least about 40% of the theoretical maximum potency of the botulinum toxin.

Embodiments of the invention also encompass a pharmaceutical composition comprising (or consisting of or consisting essentially of) a botulinum toxin, wherein the botulinum toxin is not stabilized by a protein excipient, but rather a polyethylene glycol, and a compound selected from the group of compounds consisting of a monosaccharide, a disaccharide, a trisaccharide, a metal, an alcohol, and an amino acid, wherein the potency of the botulinum toxin is at least about 20% of the theoretical maximum potency of the botulinum toxin.

Embodiments of the invention also encompass a pharmaceutical composition comprising (or consisting of or consisting essentially of) a botulinum toxin, wherein the botulinum toxin is stabilized by a polyethylene glycol, and a compound selected from the group of compounds consisting of a monosaccharide, a disaccharide, a trisaccharide, a metal, an alcohol, and an amino acid, wherein the potency of the botulinum toxin is at least about 30% of the theoretical maximum potency of the botulinum toxin.

Embodiments of the invention also encompass a pharmaceutical composition comprising (or consisting of or consisting essentially of) a botulinum toxin, wherein the botulinum toxin is stabilized by a polyethylene glycol, and a compound selected from the group of compounds consisting of a monosaccharide, a disaccharide, a trisaccharide, a metal, an alcohol, and an amino acid, wherein the potency of the botulinum toxin is at least about 40% of the theoretical maximum potency of the botulinum toxin.

Embodiments of the invention also encompass a pharmaceutical composition comprising (or consisting of or consisting essentially of) a botulinum toxin, wherein the botulinum toxin is stabilized by a polyethylene glycol, and a compound selected from the group of compounds consisting of a monosaccharide, a disaccharide, a trisaccharide, a metal, an alcohol, and an amino acid, wherein the potency of the botulinum toxin is at least about 50% of the theoretical maximum potency of the botulinum toxin.

Embodiments of the invention also encompass a pharmaceutical composition comprising (or consisting of or consisting essentially of) a botulinum toxin, wherein the botulinum toxin is stabilized by a polyethylene glycol, and a compound selected from the group of compounds consisting of a monosaccharide, a disaccharide, a trisaccharide, a metal, an alcohol, and an amino acid, wherein the potency of the botulinum toxin is at least about 60% of the theoretical maximum potency of the botulinum toxin.

Embodiments of the invention also encompass a pharmaceutical composition comprising (or consisting of or consisting essentially of) a botulinum toxin, wherein the botulinum toxin is stabilized by a polyethylene glycol, and a compound selected from the group of compounds consisting of a monosaccharide, a disaccharide, a trisaccharide, a metal, an alcohol, and an amino acid, wherein the potency of the botulinum toxin is at least about 70% of the theoretical maximum potency of the botulinum toxin.

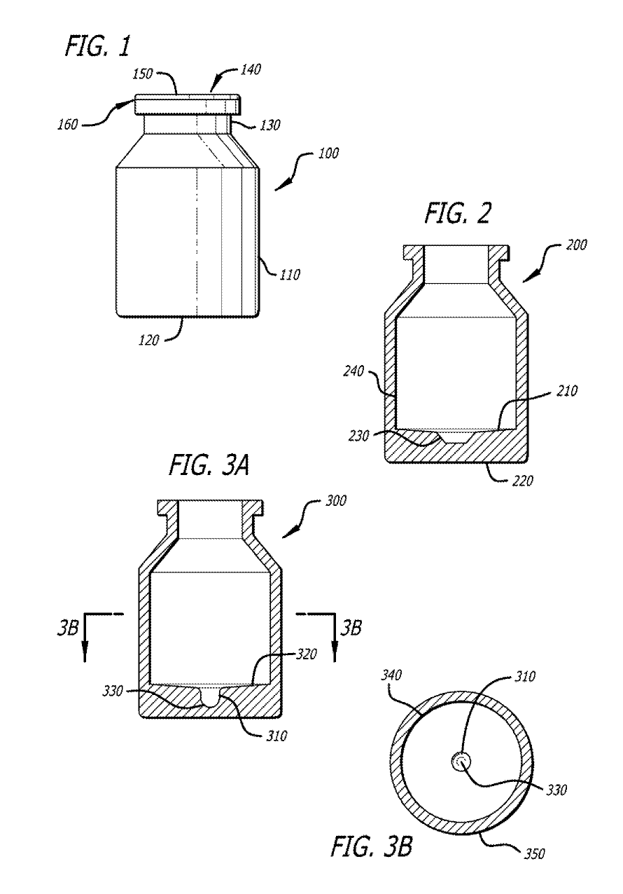

Turning to the Figures, a first embodiment of the invention is shown in FIG. 1 and designated with the reference numeral 100. The embodiment 100 includes vial wall 110 and vial base 120. Vial neck 130 provides mechanical connection between vial wall 110 and vial lip 150. Vial lip 150 is shaped to allow for secure attachment of a capping unit, such shape including a narrowing of vial lip 150 between vial lip lower edge 160 and vial lip upper edge 140.

FIG. 2 is a longitudinal cross-section of the interior of an embodiment of the invention designated with the reference numeral 200. Vial walls 240 provide mechanical connection between vial base 220 and vial neck. Vial inner bottom 210 is shallowly-angled toward vial well 230 to assist with solution collection in vial well 230. The walls of vial well 230 are angled downward to a flat-bottomed collection area.

FIG. 3A is a longitudinal cross-section of the interior of an embodiment of the invention designated with the reference numeral 300. Vial inner bottom 320 is shallowly-angled toward vial well 310 to assist with solution collection in vial well 330. The walls 310 of vial well 330 curve downward to a curved collection area.

FIG. 3B is a top-down longitudinal cross-section of an embodiment of the invention showing the outer vial wall 350 and the inner vial wall 340. The vial inner bottom is shallowly-angled toward vial well 330 to assist with solution collection in vial well 330. The walls 310 of vial well 330 curve downward to a curved collection area.

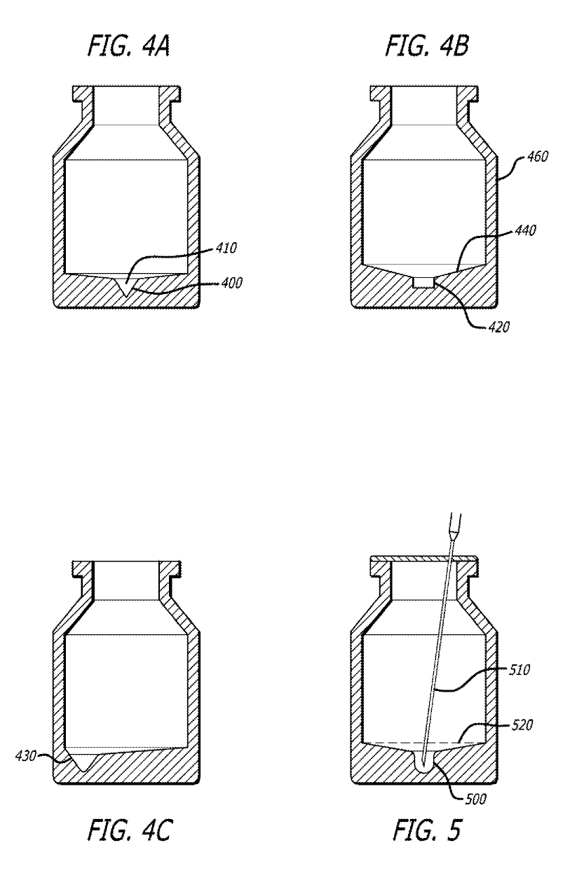

FIG. 4A is a longitudinal cross-section of the interior of an embodiment of the invention wherein the vial well 410 assumes a conical shape resulting from the angle of vial well walls 400. The vial inner bottom is shallowly-angled toward vial well 410 to assist with solution collection in vial well 410.

FIG. 4B is a longitudinal cross-section of the interior of an embodiment of the invention wherein vial well 420 is of a cubicle shape resembling a square, rectangle, or trapezoid in the cross-sectional view. Vial inner bottom 440 slopes gradually toward vial well 420 to aid in the collection and concentration of liquid material.

FIG. 4C is a longitudinal cross-section view of an embodiment of the invention wherein vial well 430 is not situated in the middle of the interior bottom of the vial, but rather adjacent to the inner vial wall. The vial inner bottom is shallowly-angled toward vial well 430 to assist with solution collection in vial well 430.

FIG. 5 is an exterior view (through the transparent material of the vial) of an embodiment of the invention wherein needle 510 is inserted through the elastomer material covering the mouth opening of the vial and the needle's tip is placed in well 500. Dashed line 520 indicates the base of the vial inner wall, which meets the vial inner bottom at an angle of greater than 90 degrees to mechanically assist in the concentration and collection of the liquid material.

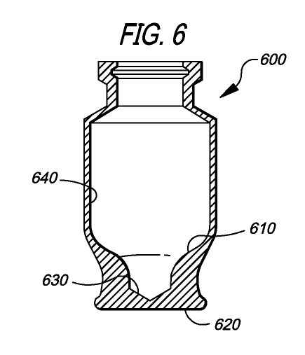

FIG. 6 is an exterior view (through the transparent material of the vial) of an embodiment of the invention designated with the reference numeral 600. Vial walls 640 provide mechanical connection between vial base 620 and vial neck. Vial inner bottom 610 is shallowly-angled toward vial well 630 to assist with solution collection in vial well 630. The walls of vial well 630 are angled downward to a V-shaped collection area.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A system for storage and subsequent handling of botulinum toxin-containing compositions, the system comprising:
a storage vessel having a top, side and base;
a well incorporated into and part of the storage vessel's base; and
a botulinum toxin-containing composition contained within the storage vessel.

2. The system of claim 1, wherein the well portion is adjacent to the inner wall of the storage vessel.

3. The system of claim 1, wherein the well portion is centered within the base portion of the storage vessel.

4. A system as in claim 2, wherein the well of the vial has a cylindrical shape with a rounded bottom.

5. A system as in claim 2, wherein the well of the vial has a conical shape.

6. A system as in claim 2, wherein the well of the vial has a cubical shape.

7. A system as in claim 2, wherein the well of the vial has a pyramidal shape.

8. A system as in claim 2, wherein the portion of the interior base of the vial surrounding the well slopes downward towards the well.

9. A system as in claim 2, wherein the well is located in the center of the interior surface of the vial's bottom.

10. A method for the storage and subsequent handling of botulinum toxin-containing compositions, the method comprising placing BTX or a BTX composition in and around the well of a vial containing a well, vacuum-drying or lyophilizing the BTX composition in the well of the vial, sealing the vial containing the vacuum-dried or lyophilized BTX composition, reconstituting the BTX composition in the vial, and extracting the reconstituted BTX composition from the well of the vial.
